(19) United States
(12) Patent Application Publication
(10) Pub. No.: US 2024/0252780 A9
(43) Pub. Date: Aug. 1, 2024

(54) SYSTEMS AND METHODS FOR IMPROVING SLEEP AND OTHER INDICATIONS, INCLUDING TREATING ANXIETY

(71) Applicants: Haven Technologies Inc., New York, NY (US); Beth Israel Deaconess Medical Center, Inc., Boston, MA (US)

(72) Inventors: Rami Burstein, Chestnut Hill, MA (US); Mark Severs, New York, NY (US); Jeff Sheely, Washington, DC (US); Ajay Shashidar Kori, Washington, DC (US)

(73) Assignees: Haven Technologies Inc., New York, NY (US); Beth Israel Deaconess Medical Center, Inc., Boston, MA (US)

(21) Appl. No.: 18/036,455

(22) PCT Filed: Nov. 12, 2021
(86) PCT No.: PCT/US2021/059123

§ 371 Date: May 11, 2023

Related U.S. Application Data
(60) Provisional application No. 63/113,294, filed on Nov. 13, 2020.
(60) Provisional application No. 63/113,299, filed on Nov. 13, 2020.

(51) Int. Cl. A61M 21/00; A61N 5/06

(52) U.S. Cl. CPC A61M 21/00; A61N 5/0613; A61M 2205/051; A61M 2210/0612; A61N 2005/0648; A61N 2005/0663

(57) ABSTRACT

The present disclosure generally relates, in certain embodiments, to light-based treatments. In some case, a subject having or at risk for a condition such as insomnia can be treated, e.g., with light, such as green light. Other conditions include sleep disorders such as sleep apnea and other indications. In some cases, the light that is administered to the subject may be green light, or the light may have a characteristic wavelength only in a wavelength range between 510 nm and 550 nm with a bandwidth no larger than 20 nm full-width-half-maximum. Other embodiments described herein are generally directed to systems for treating such conditions, kits for treating such conditions, or the like.

### RELATED APPLICATIONS

This application claims the benefit of U.S. Provisional Patent Application Ser. No. 63/113,294, filed Nov. 13, 2020, entitled “Systems and Methods for Improving Sleep and Other Indications,” by Burstein, et al., and of U.S. Provisional Patent Application Ser. No. 63/113,299, filed Nov. 13, 2020, entitled “Methods and Systems for Treating Anxiety and Other Indications,” by Burstein, et al., each of which is incorporated herein by reference in its entirety.

### FIELD

The present disclosure generally relates to light-based treatments, including but not limited to treatment using green light. The light may be applied to subjects suffering or at risk from insomnia, other sleep disorders, anxiety, or other indications.

### BACKGROUND

Light plays a critical role in the regulation of sleep and alertness. In mammals, the regulation of sleep by light is mediated mainly by blue light-sensitive retinal ganglion cells (containing the melanopsin photopigment) that through their projections to the suprachiasmatic nucleus, synchronize circadian timing with the solar system by suppressing melatonin—a hormone whose secretion by the pineal gland facilitates sleep, and whose suppression facilities wakefulness and alertness.

However, some people suffer from sleep disorders and accordingly have difficulty sleeping in general. It should be understood that while most humans will experience some degree of sleep irregularities from time to time (for example, due to the events of daily life, such as acute anxiety the night before a test in school), sleep disorders are distinct from this in that individuals suffering from such disorders often have such difficulties with proper sleep behavior that it interferes with normal daily functioning. In addition, such disorders are often not immediately connectable with the events of daily life. Accordingly, for such people suffering from sleep disorders, some intervention may be required.

Also, anxiety disorders are a group of mental disorders characterized by significant feelings of anxiety and fear. Physical symptoms of subjects suffering from an anxiety disorder can include, for example, increased heart rate and shakiness. It should be understood that although most humans experience some amounts of anxiety on a regular basis, anxiety disorders are distinct from this, in that the anxiety can be sufficiently intense in subjects suffering from such anxiety disorders as to interfere with normal daily functioning, and thus, some intervention may be required. There are a variety of different anxiety disorders, including generalized anxiety disorder, specific phobia, social anxiety disorder, separation anxiety disorder, agoraphobia, panic disorder, selective mutism, etc., and treatments for people suffering from such anxiety disorders are needed.

### SUMMARY

The present disclosure generally relates to light-based treatments, for example, for subjects suffering or at risk from insomnia, other sleep disorders, anxiety, or other indications, including those described herein. The subject matter of the present disclosure involves, in some cases, interrelated products, alternative solutions to a particular problem, and/or a plurality of different uses of one or more systems and/or articles.

One aspect is generally directed to a method comprising administering, to a subject not indicated for treatment of photophobia, green light having a characteristic wavelength only in a wavelength range between 510 nm and 550 nm with a bandwidth no larger than 20 nm full-width-half-maximum within 3 hours before the subject goes to sleep.

Another aspect is generally directed to method comprising administering, to a subject not indicated for treatment of photophobia, green light having a characteristic wavelength only in a wavelength range between 510 nm and 550 nm with a bandwidth no larger than 20 nm full-width-half-maximum after sunset.

In another aspect, the method is generally directed to administering, to a subject diagnosed with insomnia, green light having a characteristic wavelength only in a wavelength range between 510 nm and 550 nm with a bandwidth no larger than 20 nm full-width half-maximum within 3 hours before the subject goes to sleep.

Still another aspect is generally directed to a method comprising administering, to a subject suffering from jet lag, green light having a characteristic wavelength only in a wavelength range between 510 nm and 550 nm with a bandwidth no larger than 20 nm full-width half-maximum within 3 hours before the subject goes to sleep.

Yet another aspect is generally directed to administering, to a subject diagnosed with sleep apnea, green light having a characteristic wavelength only in a wavelength range between 510 nm and 550 nm with a bandwidth no larger than 20 nm full-width half-maximum within 3 hours before the subject goes to sleep.

Also, certain aspects are generally directed to methods comprising administering, to a subject not indicated for treatment of photophobia, green light having a characteristic wavelength only in a wavelength range between 510 nm and 550 nm with a bandwidth no larger than 20 nm full-width half-maximum and a luminance of no more than 100 cd/m², no more than 5 cd/m², etc.

In addition, some aspects are generally directed to administering, to a subject having an anxiety disorder, green light having a characteristic wavelength only in a wavelength range between 510 nm and 550 nm with a bandwidth no larger than 20 nm full-width half-maximum and a luminance of no more than 100 cd/m², no more than 5 cd/m², etc.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1B illustrate response curves of cones, and spectral wavelength and spectral irradiance, in accordance with some embodiments;

FIGS. 2A-2B illustrate hourly amounts of waking, REM, and non-REM sleep, in certain embodiments;

FIG. 3 illustrates daily totals for the data shown in FIGS. 2A-2B, in accordance with certain embodiments;

FIGS. 4A-4B illustrate hourly amounts of waking, REM, and non-REM sleep, in certain embodiments; and

FIG. 5 illustrates daily totals for the data shown in FIGS. 4A-4B, in accordance with certain embodiments.

### DETAILED DESCRIPTION

The present disclosure generally relates, in certain embodiments, to light-based treatments. In some case, a subject having or at risk for a condition such as insomnia, anxiety, or other conditions can be treated, e.g., with light, such as green light. Other conditions include sleep disorders such as sleep apnea and other indications. In some cases, the light that is administered to the subject may be green light, or the light may have a characteristic wavelength only in a wavelength range between 510 nm and 550 nm with a bandwidth no larger than 20 nm full-width-half-maximum. Other embodiments described herein are generally directed to systems for treating such conditions, kits for treating such conditions, or the like.

One aspect is generally directed to systems and methods for treating subjects that have trouble sleeping. In some cases, the subject may be clinical diagnosed as having a sleep disorder, including any of those described herein. The sleep disorder may be so excessive or persisting to a degree that it exceeds the normal variability in sleep patterns, e.g., in response to day-to-day events. In addition, in certain embodiments, the sleep disorder is so severe that it decreases a subject's ability to function in daily life or prevents normal functioning by the subject, for instance, as determined by a suitable clinician, such as a doctor. As discussed in detail below, light may be administered to the subject to treat or prevent the sleep disorder.

As a non-limiting example, in one set of embodiments, the subject may have or be at risk for insomnia. In some cases, the subject may have difficulty falling asleep, and/or wake up too early before getting sufficient sleep, and/or wake up often multiple times in the night, preventing sufficient sleep from occurring. Insomnia can result in daytime sleepiness, low energy, irritability, depression, trouble focusing or learning, or the like. In some cases, the insomnia may be clinically diagnosed. In some cases, a subject having insomnia cannot fall asleep for at least 5 minutes, at least 10 minutes, at least 15 minutes, at least 20 minutes, at least 30 minutes, at least 45 minutes, at least 1 hour, or more.

As another example, the subject may have or be at risk for sleep apnea. In sleep apnea, the subject experiences pauses in breathing or periods of shallow breathing more often than normal. The pauses can last from a few seconds to a few minutes, and can happen many times a night. Sleep apnea can be accompanied by loud snoring.

Other examples of sleep disorders include, but are not limited to, narcolepsy, idiopathic hypersomnia, Kleine-Levine Syndrome, sleep-wake phase disorder, irregular rhythm sleep-wake disorder, non-24 sleep-wake disorder, sleepwalking, night terrors, sleep-related sexual abnormal behavior, REM sleep behavior disorder (RSBD or RBD), sleep paralysis, nightmare disorder, sleep-related hallucinations, restless legs syndrome (RLS), Willis-Ekbom disease, periodic limb disorder (PLMS), idiopathic hypersomnia, circadian rhythm sleep disorder, or the like. As yet another example, the subject may have or be at risk for jet lag, which may be acute (experienced temporarily immediately after travel), or chronic (e.g., persisting well after the normal recovery time for jet lag), which may result in delayed sleep phase syndrome (DSPD), excessive daytime sleepiness, circadian rhythm sleep disorder, or other jet lag or temporal sleep disorders. In some cases, sleep disorders such as these can be treated as described herein. For example, a subject suffering from a disorder such as these may be treated with light, such as green light, e.g., such as described herein.

Another aspect is generally directed to systems and methods for treating subjects that have or are at risk for anxiety. Anxiety is often characterized by an unpleasant state of inner turmoil, unease, worry, etc. often accompanied by nervous behavior such as pacing back and forth, somatic complaints, rumination, muscle tension, restlessness, fatigue, lack of concentration, or the like.

For example, in certain embodiments, the present disclosure generally relates to light-based treatments. In some case, a subject having or at risk for a condition such as anxiety or other conditions can be treated, e.g., with light, such as green light. In some cases, the light that is administered to the subject may have a characteristic wavelength only in a wavelength range between 510 nm and 550 nm with a bandwidth no larger than 20 nm full-width-half-maximum. For instance, the light may be predominately green light. Other embodiments described herein are generally directed to systems for treating such conditions, kits for treating such conditions, or the like.

In some cases, the anxiety may be clinically diagnosed, e.g., the anxiety can be so severe that it decreases a subject's ability to function in daily life or prevents normal functioning by the subject, for instance, as determined by a suitable clinician, such as a doctor, a psychiatrist, a psychologist, etc. Clinical anxieties are often excessive or persisting to a degree that exceeds normal fear or anxiety. They may occur over relatively long periods of time, e.g., for several weeks or months. For instance, such anxiety symptoms may persist for 1, 2, 3, 4, 6, or more months. These anxiety disorders may include generalized anxiety disorder, specific phobia, social anxiety disorder, separation anxiety disorder, agoraphobia, panic disorder, selective mutism, or the like.

The subject is a human subject in one set of embodiments. In other embodiments, the subject may be a non-human mammal, such as a cow, sheep, goat, horse, rabbit, pig, mouse, rat, dog, cat, a primate (e.g., a monkey, a chimpanzee, etc.), or the like. The subject may be a subject having or at risk for insomnia, anxiety, or other conditions such as are described herein. In some cases, the subject is one that has a condition, such as insomnia, anxiety, or other conditions such as are described herein. In other cases, the subject is one that is at risk of having a condition, such as insomnia, anxiety, or other conditions described herein.

In one set of embodiments, the subject is a subject that is not indicated for treatment of photophobia. For example, the subject may be a subject that does not have and/or is not at risk for migraine. As other examples, the subject may be a subject that does not have and/or is not at risk for post-traumatic headache, headache caused by traumatic brain injury, retinitis pigmentosa, Leber's Congenital Amaurosis, retinal degenerative disease, achromatopsia (e.g., either totally colorblind or almost totally colorblind), albinism, night blindness, and cortical blindness. In one embodiment, the subject does not have a condition described in Int. Pat. Apl. Pub. No. WO 2016/205669.

In some embodiments, the subject may be treated by exposure to light, such as green light. For example, the exposure may the exposure of the subject's body in general (for instance, using a lamp that generally exposes most of a room to light), or a specific portion of the body of the subject (e.g., the subject's eyes or face) may be exposed to light, etc. It should be understood that the exposure to light by the subject's body may include the subject wearing ordinary clothing (e.g., sleepwear, normal daily wear, etc.), being at least partially covered by a blanket or other bedding, or the like; for example, the subject need not be naked (although they can be) and the light may not necessarily completely uniformly illuminate the subject (although it can).

The light may have a characteristic or maximum wavelength of between 490 nm and 570 nm, between 495 nm and 570 nm, between 500 nm and 565 nm, etc. In some cases, the light may have a characteristic or maximum wavelength of between 500 nm and 560 nm, or between 510 nm and 550 nm. In some cases, the characteristic or maximum wavelength may be between 515 nm and 525 nm, between 520 nm and 530 nm, or between 525 nm and 535 nm. In certain embodiments, the characteristic or maximum wavelength may be around 520 nm, around 525 nm, or around 530 nm, e.g., within +/−5% or +/−10% of these values. Such light including any of these may be monochromatic, or have a bandwidth no larger than 50 nm, no larger than 40 nm, no larger than 30 nm, no larger than 20 nm, or no larger than 10 nm, as determined at full-width half-maximum (FWHM). The characteristic wavelength of the light may be determined as the weighted average of a spectrum of light wavelengths, i.e., weighted based on their intensities within the spectrum. As discussed herein, such wavelengths may be produced by a light source, and/or light may be passed through a filter to produce such wavelengths. Without wishing to be bound by any theory, it is believed that the light administered to the subject need not be of a single or narrow frequency (although it can be), but may be of any light suitable for predominately activating the green cones within the eye. Thus, some “leakage” of light, e.g., at wavelengths that causes some stimulation of red or blue cones, may not be detrimental to the administration of green light to the subject.

The subject may be exposed to the light for any suitable length of time, e.g., for treatment. For instance, the subject may be exposed to the light for at least 1 minute, at least minutes, at least 10 minutes, at least 15 minutes, at least 20 minutes, at least 30 minutes, at least 45 minutes, at least 1 hour, at least 2 hours, at least 3 hours, at least 4 hours, at least 6 hours, at least 9 hours, or at least 12 hours. In addition, in some cases, the subject may be exposed to the light for no more than 12 hours, no more than 9 hours, no more than 6 hours, no more than 4 hours, no more than 3 hours, no more than 2 hours, no more than 1 hour, no more than 45 minutes, no more than 30 minutes, no more than 20 minutes, no more than 15 minutes, no more than 10 minutes, no more than 5 minutes, or no more than 1 minute. In some cases, the subject may be exposed to the light for combinations of any of these times; for instance, the subject may be exposed to the light for a period of time between 10 minutes and 1 hour, between 1 hour and 2 hours, between 1 hour and 3 hours, or the like. The exposure may be provided once, or in 2, 3, 4, or more sessions per day in order to treat the subject.

In some embodiments, the light may have a luminance (luminous intensity per unit area) on the subject (e.g., on the subject's eyes) of less than 70 cd/m². Luminance is perceived in humans as “brightness.” In certain embodiments, the luminance may be less than 100 cd/m 2 or less than 75 cd/m². In some cases, the luminance may be less than 50 cd/m², less than 30 cd/m², less than 25 cd/m², less than 20 cd/m², less than 15 cd/m², less than cd/m², etc. In some cases, only a very low luminance is needed, e.g., less than 5 cd/m², less than 3 cd/m², less than 1 cd/m², less than 0.5 cd/m², etc. In some cases, the luminance of the light on the subject may also be at least 0.5 cd/m², at least 1 cd/m², at least 2 cd/m², at least 3 cd/m², at least 5 cd/m², etc. In some cases, the luminance may be between any combination of these, e.g., between 0.5 cd/m² and 5 cd/m², between 0.5 cd/m² and 1 cd/m², between 1 cd/m² and 5 cd/m², or the like.

In certain cases, the light may have an illuminance (luminous flux per unit area) on the subject (e.g., on the subject's eyes) of less than 70 lux. Luminance is perceived in humans as light intensity. In some cases, the illuminance may be less than 50 lux, less than lux, less than 20 lux, less than 15 lux, etc. In some cases, only a very low illuminance is needed, e.g., less than 10 lux. In some cases, even lower illuminance can be used, e.g., less than 5 lux, less than 3 lux, less than 1 lux, less than 0.5 lux, etc. In some cases, the illuminance of the light on the subject may also be at least 0.5 lux, at least 1 lux, at least 2 lux, at least 3 lux, at least 5 lux, etc. In some cases, the illuminance may be between any combination of these, e.g., between 0.5 lux and 5 lux, between 0.5 lux and 1 lux, between 1 lux and 5 lux, or the like.

The light administered to the subject may be administered using any suitable light source. In some cases, the light source may be one that specifically produces light, e.g., green light, having a characteristic wavelength only in a wavelength range between 510 nm and 550 nm with a bandwidth no larger than 20 nm full-width half-maximum, or other characteristic wavelengths such as are discussed herein. Devices able to produce such green light may be obtained commercially in some cases.

In certain cases, white light sources or other types of light sources (e.g., video screens, the sun, etc.) can be used. However, they typically produce a range of colors, and thus do not inherently produce green light, or light having a characteristic wavelength only in the above wavelengths. In some embodiments, such light sources may be modified in some fashion to produce light having desirable wavelengths as discussed herein. For example, a suitable filter may be used to screen out or reduce undesirable wavelengths, thereby causing the resulting light passing through the filter to have the desirable wavelengths. Many such filters can be obtained commercially. One or more such filters can be used. The light may be filtered such that the light passing through the filter has a characteristic or maximum wavelength of between 490 nm and 570 nm, between 495 nm and 570 nm, between 500 nm and 565 nm, etc., or other characteristic or maximum wavelengths such as any of those described herein. As another example, the video screen may be controlled (e.g., by a computer) to produce light having desirable wavelengths such as are described herein. For instance, the video screen may only produce images formed from green light. Accordingly, in some embodiments, the device may not necessarily be a device that can only produce green light.

The light may be administered to the subject from one light source, or two, three, or more light sources. If more than one light source is used, the light sources may be the same or different. Examples of light sources include, but are not limited to, light bulbs (e.g., incandescent or fluorescent, light emitting diodes (LEDs), laser diodes, lasers, etc. Other examples include of light sources include fluorescent, incandescent, laser source, frequency-doubled laser source, halogen, xenon, tungsten halogen, phosphorescent, or photo-luminescent light source, any combination thereof, or any other type of light source. Other non-limiting examples of suitable light sources can be found in Int. Pat. Apl. Pub. No. WO 2016/205669, incorporated herein by reference in its entirety for all purposes. The light source itself may only produce light of the characteristics described herein (e.g., green light), and/or the light source may produce light at a variety of wavelengths (e.g., white light) that are then filtered or removed to produce light of the characteristics described herein.

The light source may, in some cases, be controlled to produce the illuminances as described herein (e.g., by controlling the intensity of light produced by the light source, the distance between the subject and the source of light, etc.), and/or be controlled to produce light for times such as those described herein. For example, a light souce may be dimmed by reducing the amount of power or current applied to it, by controlling the number of cycles the light source is on/off, or the like. Those of ordinary skill in the art will be familiar with systems and methods to control a light source to produce light of a desired intensity, duration, etc.

In some cases, at least 50% of the light reaching the subject (e.g., reaching the subject's eyes, or other portions of the subject) is light having the characteristics described herein, e.g., green light, and/or light having a characteristic wavelength of between 490 nm and 570 nm, or other wavelengths such as those described herein. In addition, in certain embodiments, at least 60%, at least 70%, at least 80%, at least 90%, at least 95%, or at least 99% of the light reaching the subject is green light, and/or light having the characteristics described herein. The light reaching the subject may be produced by one or more light sources such as described herein, although in some cases, there may also be extraneous light from other sources that is present (for example, stray light passing through a crack in a doorway).

In one set of embodiments, the light reaching the subject is determined at the location of the subject's eyes. For instance, the light reaching the subject's eyes may have the characteristics described herein, e.g., green light, and/or light having a characteristic wavelength of between 490 nm and 570 nm, or other wavelengths such as those described herein. Light reaching other parts of the subject may or may not have the same characteristics. For example, the subject may be positioned such that a light source having such wavelengths is directed specifically to the subject's eyes (or face) (e.g., as opposed to being used as an ambient light source within the room), or the subject may wear a filter that allows only desired wavelengths to reach the eyes (for example, glasses or sunglasses that are able to filter other wavelengths of light), etc. As another non-limiting example, the light source may include one or more filters that can be used to produce the desired wavelengths, e.g., attached to the light source.

The amount of extraneous light reaching the subject may be minimized in accordance with certain embodiments. For example, the subject may be positioned in a darkened room with no active light sources (e.g., that are actively producing light that reaches the subject) other than the ones described herein, e.g., producing light of the characteristics described herein. For instance, other lights within the room may be dimmed, turned off, covered, deactivated, etc. In some cases, and the subject may be positioned in a room that has no external windows, the windows in the room may be covered, the subject may be administered at night or after sunset, etc., thereby removing the sun or external lights as potential light sources.

In addition, in some cases, during administration, the subject may refrain from using a cellphone or a computer, television, wristwatch, or other device that may act as a light source, e.g., by producing light via a video screen other than light having the characteristics described herein. Such devices having a screen may not be present in the room, and/or may be present, but at least the video screens are turned off or dimmed. However, in some embodiments, such device may be active, but configured to produce only light having the characteristics described herein, e.g., via hardware and/or software (e.g., the video screen may enter a mode such as a “green mode” where only certain wavelengths of light are displayed, such as the wavelengths described herein). Accordingly, in such cases, the device may act as a light source such as is described herein, e.g., one that is able to administer suitable light to the subject (e.g., green light, and/or light having a characteristic wavelength such as described herein), alone and/or in conjunction with other light sources such as those described herein.

The light may be administered to the subject at any suitable time. As non-limiting examples, the light may be administered to the subject after sunset, or within a certain amount of time before the subject goes to bed or to sleep. For example, the light may be applied within at least 10 minutes, within at least 15 minutes, within at least 30 minutes, within at least 45 minutes, within at least 1 hour, within at least 2 hours, within at least 3 hours, etc., of the subject going to bed or going to sleep.

In one embodiments, the light may be continued to be administered, even after the subject goes to sleep. However, in other embodiments, the administration of the light may be altered after the subject goes to the sleep. When a subject goes to sleep may be determined, for example, by another person, or automatically, for instance, by using noise sensors, motion sensors, etc., to determine when the subject has stopped moving and appears to be sleeping. After the subject goes to sleep (e.g., as determined by such sensors), for example, the light may be deactivated or extinguished. However, in some cases, the light may continue to be administered. In addition, in certain embodiments, the light may be dimmed after the subject goes to sleep. For instance, the light administered to the subject may be dimmed to less than 100 cm/m², less than 75 cd/m², less than 50 cd/m², less than 25 cd/m² less than 10 cd/m², less than 5 cd/m², less than 3 cd/m², less than 1 cm/m², or less than 0.5 cd/m². In some cases, the light may be dimmed to less than 5 lux, less than 3 lux, less than 1 lux, or less than lux.

In some embodiments, the light (which may optionally have been dimmed, as noted above) may be deactivated a certain time after the subject has gone to sleep. For instance, the light may be deactivated after 5 minutes, after 10 minutes, after 15 minutes, after 30 minutes, after 45 minutes, after 1 hour, after 2 hours, after 3 hours, etc., after the subject has gone to sleep.

Int. Pat. Apl. Pub. No. WO 2016/205669 is incorporated herein by reference in its entirety. In addition, U.S. Provisional Patent Application Ser. No. 63/113,294, filed Nov. 13, 2020, entitled “Systems and Methods for Improving Sleep and Other Indications,” by Burstein, et al., and U.S. Provisional Patent Application Ser. No. 63/113,299, filed Nov. 13, 2020, entitled “Methods and Systems for Treating Anxiety and Other Indications,” by Burstein, et al., are each incorporated herein by reference in their entireties.

The following examples are intended to illustrate certain embodiments of the present disclosure, but do not exemplify the full scope of the disclosure.

### Example 1

This example illustrates what narrow-band green light (NbGL) effects would be on the psychophysical findings and the cortical responses, for exposure times extended to hours rather than minutes or milliseconds. Prolonged (12 h) exposure to NbGL attenuates cortical excitability and alters patterns of sleep, NREM and REM sleep (compared to regular white light or complete darkness) were tested. The preliminary evidence for NbGL overall ability to increase total sleep time, including both NREM and REM sleep, when replacing the light (sleep) phase but not the dark (wake) phase.

For the lay person and treating physician, the idea that NbGL can improve sleep quality by decreasing the time it takes to fall asleep and increasing the amount of time sleep is maintained, as well as the amount of time sleep reaches NREM stages 3 and 4 or REM, may sound like a far-fetched scenario. However, understanding the NbGL effects on the cortex's ability to reach late-stage NREM and REM sleep may allow patients and doctors to consider this non-invasive, risk-free, and affordable light therapy as a complementary treatment that may help those suffering insomnia and daytime sleepiness. This significance is further supported by the well-known phenomenon that benzodiazepines, the most commonly prescribed sleep medications, increase stage 2 sleep (dominated by theta waves and sleep spindles) and decrease stages 3, 4 and REM sleep, potentially accounting for daytime drowsiness after apparent long sleep nights.

Light therapy is a rapidly-growing field in alternative medicine as it introduces non-invasive and safe ways to treat human diseases without using chemicals that often carry numerous unwanted side effects and risks. Factors such as timing (day-time vs. night-time exposure), duration (2 h vs. 12 h), extent (effects on sleep stages, NREM vs. REM sleep, delta power during NREM) and limitations of NbGL ability to reduce cortical excitability and improve sleep may support the current notion that environmentally-induced factors (such as blue-light emitting electronic devices) that lead to overall cortical hyperexcitability that is perilous to sleep could be solved by replacing the blue light with new and improved light therapies that dynamically manipulate color, duration and timing of exposure.

It is believed that the therapeutic NbGL effects on sleep are achieved in part through attenuation of cortical hyperexcitability that facilitates sleep onset and improved sleep quality by ‘allowing’ the cortex to spend more time in the deep NREM sleep stages and REM. Overview of experimental design and rationale for regular and dim light conditions.

All experiments will be done blinded to experimental group, both during data collection and data analysis. In all three aims, sleep-waking behavior will be quantified by continuous recording of EEG/EMG (polysomnography) over a 5-day baseline period and a 2-day experimental period. During the 5-day baseline period, all experimental groups start with a uniform 5-day period of baseline recording carried out under a “standard” daily 12 h:12 h light-dark cycle (regular 100 lux white light during the light phase/(0 lux during the dark phase), prior to the start of the 2-day period of experimental manipulations of the light conditions. During the 2-day experimental period, each group is exposed to NbGL for one portion of the 12 h:12 h light-dark cycle. The period of NbGL exposure is during one of four periods: the light period, the dark period, the last 2 h of the light period, or the last 2 h of the dark period. White light (WL) and NbGL are tested at two intensities: 100 lux and 5 lux.

Note on the representation of light-dark periods. A convention is adopted in which the alternating phases of the light-dark cycle are shown with either the light phase first or the dark phase first, depending on the experimental group. This is for the purposes of illustrating a point about the data analysis. The actual timing is the same in all groups (5 PM lights off, lights on). This change is made in the figures because of an important point regarding the analysis of the data. For those groups that receive NbGL exposure during the “dark” phase, both of the two light phases that are included in the 2-day experimental period will be light phases that follow a dark phase that had NbGL exposure. (i.e., the first of the two light phases should not be a light phase that follows the last dark phase of the baseline period.)

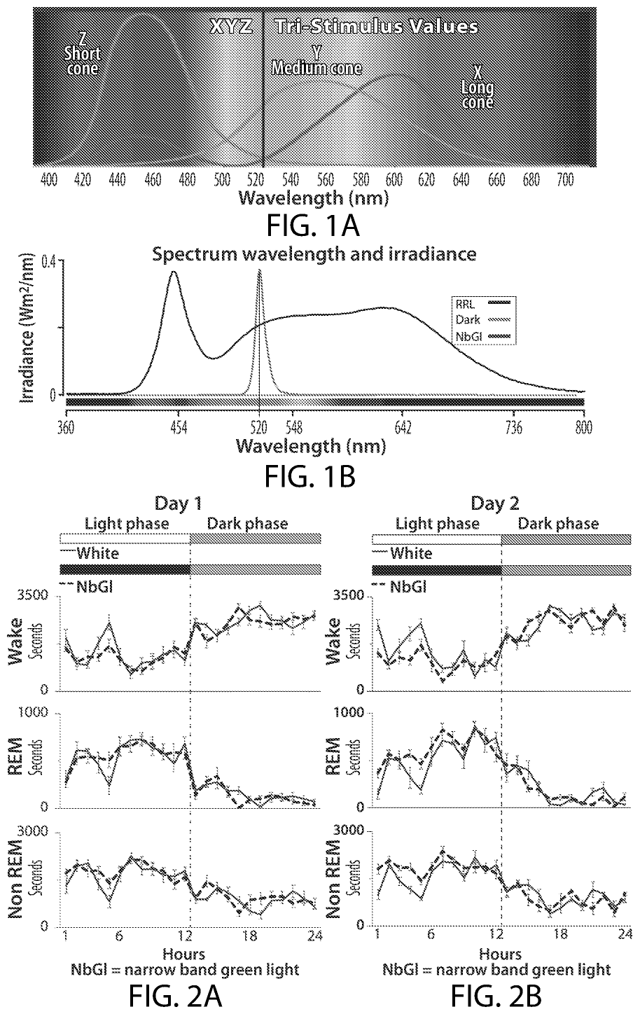

Choice of light intensities. 100 lux was selected as the regular light and 5 lux as a dim light condition because these intensities replicate the most commonly used regular and dim light conditions in studies on how circadian rhythms and sleep-wake behaviors are affected by the introduction of regular and dim light conditions during the light and dark phases. While the sources of standard and dim light conditions can be as high as 150-200 or 25-50 lux, respectively, the typical intensity that reaches the retina (after being reflected from nearby surfaces) is about 100 lux (at standard conditions) and 3-8 lux (at dim conditions). To verify that the intensity of light (white and NbGL) is equal, repeated calibrations will be performed with the International Light Technologies photometer (ILT-1700) and the Spectral Light Meter (MSC15, Gigahertz-Optik GmbH). While the spectral Light meter generates light intensity values in lux (FIG. 1B), the ILT-1700 photometer provides numerical values of luminance (intensity) in cd/m².

FIG. 1A: Response curves of blue (short), green (medium) and red (long) cones. FIG. 1B: Spectral wavelength and spectral irradiance of RRL, NbGL (generated by luminance of 100 cd/m²) and dark showing how different are these 3 conditions. Spectral irradiance is the energy emitted, reflected, transmitted or received, per unit time per nanometer. Note: Spectral irradiance is often confused with light intensity. Light intensity is expressed using luminance scale. Luminance is an indicator of how bright the light appears.

Rationale for using the 520 nm (peak) NbGL: The clinical advantage of using light that is the least sensitive to short and long wavelength cones and most sensitive to medium wavelength cones is recognized. Because short wave cones least sensitivity is at 560 nm, long wave cones least sensitivity is at 510 nm, and medium cones maximal sensitivity is around 534 nm (FIG. 1A), it can be concluded that a narrow band of light with peak value of 520 nm and a range that allows 90% of the light to be distributed within 10 nm on each side (510-530 nm), would give the best chance to establish a photic stimulus that stimulates medium cones maximally and long and short cones minimally (i.e. maximizing ratios of medium-to-long, and medium-to-short activation) (FIG. 1B).

### Example 2

This example was used to determine whether and how REM and NREM sleep duration, sleep latency, and fragmentation, are altered in rats in which the 12 h light phase (100 lux white light) of the standard light-dark cycle is replaced with 12 hrs of regular (100 lux) or dim (5 lux) NbGL.

NbGL has the ability to reduce sensitivity to light and cortical responses to visual stimuli, as well as patients' impression that it may ease their falling asleep are puzzling, and if not studied further may end up being considered as far-fetched claims. These studies will determine whether it is possible to reduce latency for sleep onset and increase sleep duration, for both NREM and REM sleep by replacing the 12 h light period (100 lux white light) of the standard 12 h:12 h light-dark cycle with 12 h NbGL of regular (100 lux) or dim (5 lux) intensity. There are several rationales that contribute to the design of a study in which the light conditions are altered during the light phase of the sleep/wake cycle. Although rats are nocturnal (meaning that most of their hunting/eating and social activities take place in the dark of the night and most of their sleep happens in the light of the day), it is well-known that like in all mammals, light plays a critical role in maintaining their circadian rhythms, normal sleep behavior, vigilance state, and food intake, and that alterations of intensity and color at inappropriate times can disrupt their locomotor activity, sleep-wake cycle, plasma melatonin, food intake and other neurological functions. Relevant to this is the puzzling observation that when effects of color of light were tested in preclinical studies (where the different lights were administered during the light phase), they included blue, amber and red but not green light. Preliminary pre-clinical observations in which 12 h exposure to NbGL replaced the light phase of the 12 h:12 h light-dark cycle. This is justified for 2 reasons: the first is the possibility that it will allow determination of whether it is possible to improve sleep or any of its aspects (latency, duration, frequency of awakenings) by replacing the light phase of the light-dark cycle and the second is the opportunity to learn whether and how NbGL modulates cortical excitability, and alters cortical characteristics of NREM and REM sleep (when replacing the light, rather than the dark phase). A detailed explanation for how these findings will be translated to humans is provided after Data Interpretation.

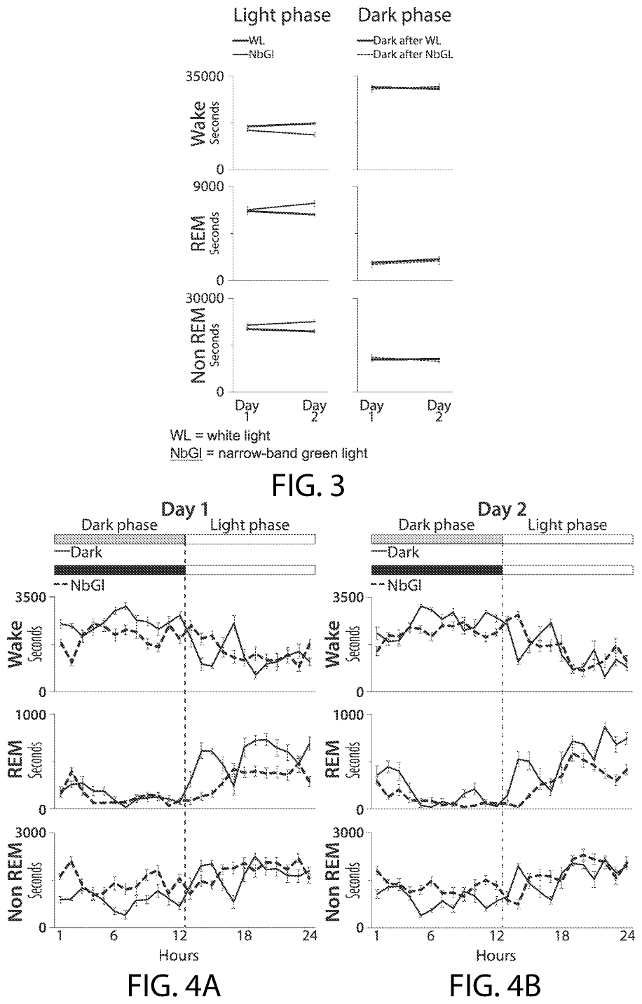

NbGL during the light phase was found to produce an increase in NREM and REM sleep. Polysomnographic recording (EEG and EMG) was carried out in female rats (n=7) maintained under a standard 12 h:12 h light-dark cycle for 7 days. The regular white light (100 lux) was then replaced by regular NbGL (100 lux) for 2 days. The dark phase (0 lux) was unchanged. The plots in FIG. 2 show the hourly amounts of waking, REM, and non-REM sleep during the last two days of the baseline period (solid black lines) and the two days of the experimental period (dashed lines). (FIG. 2A shows Day 1 and FIG. 2b shows Day 2.) Under NbGL, rats exhibited an increase in NREM and REM sleep, and a decrease in waking, compared to the time under white light, during the 12 h light phase. This effect was most pronounced on the second day of NbGL illumination (FIG. 2B, and FIG. 3, Day 2). The hourly plots shows that there is a “smoothing” of the curves under NbGL: whereas the curves show a distinct up-and-down shape under white light, with 2-3 alternating periods of greater and lesser amounts of sleep over the course of the 12 h light period, this up-and-down alternation is largely evened out during NbGL illumination. In contrast to these changes during the light phase, the curves are relatively similar during the dark phase. Thus, in this experiment, there appears to be no marked residual effect after the end of NbGL exposure. FIG. 3 shows daily totals for data in FIG. 2.

These preliminary findings suggest that 1) NbGL may exert effects on cortical activity that tend to increase both phases of sleep; 2) this effect may become more pronounced when the NbGL is maintained for more than one day; 3) this effect is most apparent during those hours of the light phase when the rats tend to show more waking with white light illumination, i.e. the NbGL tends to “smooth out” the curve and produce a more steady, maintained time course of sleep during the light phase; and 4) there appears to be no residual effect during the dark phase that follows the 12 h NGl exposure.

### Example 3

Some experiments were used to determine whether and how REM and NREM duration, sleep latency and fragmentation are altered in rats in which the 12 h dark phase of the standard light-dark cycle is replaced with 12 h of regular (100 lux) (2a) or dim (5 lux) (2b) NbGL. Since color adds excitement and emotions to our life and because certain colors are associated with happiness and well-being, it may not be desired to replace the entire 12 h light phase of the light-dark cycle with a monochromatic green light—even if it may improve sleep, calm the cortex, and increase NREM and REM sleep. Theoretically, it may be more beneficial to replace the dark phase of the light-shorten sleep latency and increase sleep duration in male and female rats by replacing the 12 h dark (0 lux) period of the regular 12 h:12 h light-dark cycle with 12 h of dim (5 lux) NbGL. While designing these studies, the following were considered: (a) in humans, exposure to dim bedroom white light is associated with insomnia and decreased sleep depth, (b) in nocturnal rodents, exposure to dim white light during the dark phase decreases sleep during the light phase, (c) when bright red light was used during the entire dark phase, it suppressed melatonin for the entire 24 h period, and (d) the effects of replacing the entire 12 h dark phase with dim green light were never studied in rodents or humans. What justifies studying effects of replacing the dark phase with dim NbGL are (1) patients' frequent asking if they could use or expect benefit from sleeping all night under dim NbGL, and (2) the absolute lack of knowledge on the most basic effects of replacing the dark phase with dim NbGL on the different aspects of sleep. A detailed explanation for how findings in this aim will be translated to humans is provided after the Data Interpretation section (at end of Research Plan).

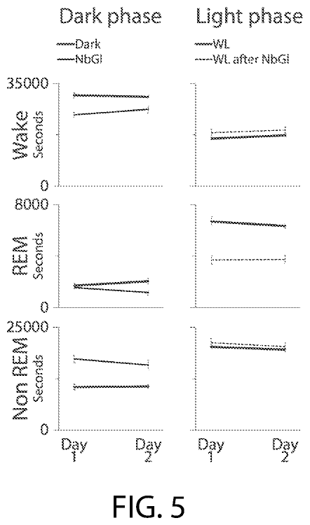

It was found that standard NbGL during the dark phase alters the normal circadian pattern by causing a reduction in the amount of sleep during the initial 3 hours of the subsequent light phase. As above, female rats (n=7) were first maintained on a 12 h:12 h light-dark cycle using regular white light (100 lux). During the 2-day experimental period, they were exposed to regular NbGL (100 lux) illumination during the dark phase, rather than during the light phase as in Aim 1; thus, the rats were exposed to 12 h NbGL (“dark” phase) followed by 12 h white light (light phase), during the 2-day experimental period. The most noticeable effect of this paradigm was apparent not during the phase of the NbGL exposure itself (during the animal's “dark” phase), but during the standard white light phase (in white light) that followed the 12 h NbGL exposure: there was a decrease in the amount of sleep during the initial 3 hours of the light phase (FIG. 4). (FIG. 4A shows Day 1 and FIG. 4b shows Day 2.) Normally, under a standard 12 h:12 h light-dark cycle with white light, the onset of the light phase initiates a progressive increase in the hourly amount of sleep, reaching a maximum after about 3 hours (FIG. 4, solid black line). In the rats exposed to NbGL during the preceding dark period, the increase in sleep at the onset of the light period appears to be delayed by about 3 hours (FIG. 4, dashed line). These preliminary data suggest that NbGL during the “dark” phase may have effects during the following light phase, and may alter the normal circadian rhythm of the sleep-waking cycle. This raises the possibility that NbGL at the wrong time may be harmful rather than helpful, as noted in the background above. In order to address this issue, NbGL will also be tested at a much lower intensity (“dim” NbGL, lux), to investigate whether such potentially harmful effects on circadian rhythmicity can be minimized while still maintaining the beneficial effects of NbGL. FIG. 5 shows the daily totals for the data in FIG. 4.

### Example 4

This example discusses data interpretation of the above examples.

General principles: Comparison of rats exposed to NbGL vs white light at the same intensity will be evidence of specific effects of the color of the light. Comparison of rats exposed to different intensities (regular NbGL vs dim NbGL, regular white vs dim white) will be evidence of effects of light intensity.

Cortical activity. Effects of NbGL on any parameters of sleep-waking behavior (total time, number of bouts, mean bout duration, for each of the three states) will be evidence of the ability of NbGL to modulate cortical activity.

Sleep Behavior. Sleep parameters will be interpreted in relation to signs of improved sleep. Changes that will be interpreted as signs of increased sleep quality are: (a) extended total sleep time, (b) sleep latency, (c) decreased number of awakenings per hour of sleep (“arousal index”, a measure of sleep fragmentation), (d) increased delta power during NREM sleep (reflecting depth or intensity of NREM sleep), and (e) increase in amount of REM.

Reasoning: (1) extended total sleep time will be interpreted as improved sleep quality because ability to sleep longer hours is thought critical for maintaining healthy brain and body, (2) short latency for sleep onset latency will be interpreted as improved sleep quality because difficulty falling asleep commonly stresses people, (3) Fewer wake episodes during the sleep phase will be interpreted as improved sleep quality because frequent waking during the night can reduce ability to reach the deepest level of sleep and cause anxiety associated with inability to fall asleep again.

NREM and REM sleep: Separate measurement of time spent in non-REM sleep and REM sleep allows further evaluation of effects on the quality of sleep and the changes induced by experimental manipulations. REM sleep occurs in discrete, relatively short bouts.

Entry into REM sleep can only occur after first reaching a state of deep NREM sleep (especially high delta power). REM sleep increases towards the later part of the sleep cycle. A relative absence of REM sleep, relative to the amount of non-REM sleep, may be a sign of less beneficial sleep. For example, the sleep induced by benzodiazepines is a lighter sleep with a relative lack of REM sleep.

Interpretation of results obtained when replacing the standard 12 h white light phase with 12 h regular NbGL: If NbGL improves sleep, it will be interpreted as suggesting that regular NbGL can modulate cortical activity to the extent that is sufficient to increase REM and NREM sleep. If it does not improve sleep, it will be interpreted as suggesting that NbGL alone is not sufficient to modulate cortical activity to the extent required to increase sleep or to trigger activation of other brain mechanisms involved in the regulation of sleep.

Interpretation of results obtained when replacing the standard 12 h white light phase with 12 h dim NbGL: If dim NbGL improves sleep, this will show that dim NBGL can modulate cortical activity to an extent that is sufficient to increase REM and NREM sleep. If regular NbGL improves sleep but dim NbGL does not improve sleep, this will show that the NbGL intensity must be maintained at the higher level to achieve the effects on cortical activity and sleep. If the effects of dim NbGL are similar to the effects of dim white, it will be concluded that at low intensity the color plays no role in cortical modulation.

Interpretations of effects seen during the dark period that follows the 12 h light period of exposure to NgBL: If wake time or other parameters do not differ between NbGL and white light in the 12 h dark period following the period of exposure to NbGL vs white light, it will be concluded that the NbGL did not have lasting effects (into the dark cycle). Conversely, if there are changes during the following dark phase, it will be concluded that the light conditions did have lasting effects. If wake time is increased during the dark period, this will support the benefits of using NbGL during sleep. If wake time is decreased, this will question the benefit of using the NbGL during sleep phase as it could represent a decreased degree of alertness on the day following night time use of NbGL.

Interpretation of results obtained when replacing the standard dark phase with NbGL: If exposure to NbGL during the normal “dark” phase decreases the amount of sleep during the following light phase, or delays the onset of sleep following the start of the light phase, this will be considered a sign of potentially harmful effects, and will question the benefit of using the NbGL during the dark phase. If such effects are reduced or eliminated in experiments where the NbGL is used at a lower intensity or used for only a restricted period at the end of the dark phase (regular or dim NbGL), then this will support the benefits of using NbGL during waking.

Harmful effects. It is possible that NbGL exposure at specific times of the light-dark cycle can produce effects that would be considered harmful, such as (1) reduction in sleep during the normal sleep phase, (2) reduction in waking during the normal waking phase, and (3) disturbance or alteration in the normal circadian rhythmicity of the sleep-waking cycle. Ability to detect such unwanted changes will allow identification of times during the light-dark cycle in which specific light conditions (color, intensity) should be avoided.

Difficulty of translation to effects in humans from studies of nocturnal animals: Ultimately, the human application that is envisioned for NbGL (with respect to sleep) is the enhancement of sleep by the use of NbGL during some limited portion of their waking hours. (i.e. it is not practical/desirable for people to spend the entire day in NbGL, or keeping it on throughout the night while sleeping.) However, to get pre-clinical information relevant to this goal, with regard to both potential helpful and harmful effects, it may be necessary to examine effects of NbGL during both the light phase and the dark phase of the rats' light-dark cycle. Exposure during the rat's dark phase is in some sense analogous to the daytime in humans, but in rats, there is an additional factor that the NbGL light is potentially disruptive to the rat's circadian rhythm. Exposure during the rat's light phase might be considered analogous to nighttime in humans, but unlike humans who (ideally) sleep throughout the night, rats are normally awake for a considerable portion (25-35%) of their light phase (and also, rats are asleep for 25-35% of their dark phase). Thus, because rats are nocturnal, and also because they show substantial periods of both sleep and waking in both phases of the light cycle, there is no single paradigm in rats that is entirely analogous to daytime effects in humans. In spite of this fundamental difficulty in interpreting the effects for their relevance in humans, it is believed that the findings in rats will provide an essential foundation for rational design of studies of NbGL effects in humans.

### Example 5

A survey was sent to purchasers of a lamp producing green light such as discussed herein. It was found that out of 171 respondents, 40.1% of respondents believed that the light was able to help them fall asleep more easily. In addition, 78.4% of respondents believed that the light was soothing, calming, and relaxing, and 58.2% of respondents believed that the light was able to decrease anxiety and stress.

### Example 6

This example illustrates a psychotherapeutic approach for the treatment of generalized anxiety disorder (GAD) improves outcome when delivered under specific (narrow band) green light condition, in accordance with another embodiment. In this example, experiments were conducted to evaluate narrow-band green light (nbGL) effects on 45-min long session for the treatment of GAD by psychotherapists.

Studies in migraine patients revealed that during attacks, exposure to blue, red, amber, and white lights trigger negative emotions (e.g., feeling more stressed and upset), and that in contrast, exposure to nbGL induces positive emotions (e.g., feeling relaxed and calm). These finding raised the possibility that under this unique light condition, psychotherapy sessions for GAD could be more productive.

The methodology involved within-subject (repeated measures) study design comparing effects of conducting 2 psychotherapy sessions under white light (WL) and 6 sessions under nbGL (delivered by Allay Lamp, Allay, USA) on patients' self-evaluation of treatment using the State part (Y-1, Qs.1-20) of the State-Trait-Anxiety-Inventory questionnaire at the beginning and end of each session. Included in the analysis were 7 male and 6 female patients (18-72 y/o) diagnosed with GAD.

Results were as follows. (a) Total score. WL=42.5 (41-48.2) [median (IQR)] before vs. 42.5 (38-49.2) after (p=0.37). nbGL=40.8 (37.3-46.5) before vs. 39.1 (37.6-45.5) after (p=0.70). (b) Reversed scored items (symptom-positively worded). WL=2 (1-2.5) before vs. 2 (1.5-2.5) after (p=0.60). nbGL=2 (1.3-2.5) before vs. 2.1 (1.5-2.6) after (p=0.007). (c) State anxiety-present items (symptom-negatively worded) WL=2.5 (1.5-3.5) before vs. 2.2 (1.5-3) after (p=0.35). nbGL=2 (1.3-2.8) before vs. 1.8 (1.3-2.6) after (p=0.001) (Wilcoxon rank sum test). Comparing between and within group analysis of the 4 conditions as independent variables using ANOVA and Post-hoc analysis for multiple comparison (Tukey HSD) revealed the following: (a) symptom-positively worded−differences between after nbGL vs. the other 3 variables were significant [ANOVA F=5.69, p=0.0008; Tukey HSD p<0.01). (b) symptom-negatively worded−differences between after nbGL vs. the other 3 variables were significant [ANOVA F=20.4, p=0.0002; Tukey HSD p<0.001).

In conclusion, it was found that using nbGL as an add-on to psychotherapy improves patients' state of anxiety.

### Example 7

This example illustrates that prolonged exposure to narrow band green light (nbGL) facilitates sleep onset, in accordance with one embodiment. In this example, experiments were conducted to determine whether sleep behavior can be improved by daily exposure to nbGL in the 2 hrs period that precede bedtime.

Light plays an important role in the regulation of sleep and alertness. In mammals, the regulation of sleep by light is mediated mainly by blue light-sensitive retinal ganglion cells that through their projections to the suprachiasmatic nucleus, synchronize circadian timing with the solar system by suppressing melatonin—a hormone whose secretion by the pineal gland facilitates sleep, and whose suppression facilities wakefulness and alertness. In contrast, low-irradiance green light promotes rapid sleep onset and longer sleep duration, potentially through a color-spectrum unique activation of the sleep-promoting neurons in the ventrolateral preoptic area.

The methodology involved within-subject (repeated measures) study design comparing effects of 2 hrs-before-bedtime exposure to white light (WL) vs. nbGL (delivered by Allay Lamp, Allay, USA) on sleep latency, awakening episodes total awake time, and total sleep time—using a daily sleep diary for 60 days (30 days under each light condition). Included in the analysis were 12 subjects (8 males, 4 females, between 61-81 y/o) with no known sleep disorder or use of over-the-counter or prescribed sleeping aids. Throughout the study, participants were instructed to refrain from using computers and cell phones or watching TV.

The results were as follows. Compared to WL, exposure to nbGL reduced sleep latency by 37.5% [508 min (391-598) vs. 318 min (150-471) median (IQR); p=0.022], number of awakening episodes by 32% [63 (34-72) vs. 43 (23-68); p=0.018], and total awake time by 35.4% [618 min (296-983) vs. 399 min (115-729); p=0.049], and consequently had the tendency to marginally increase total sleep time (p=0.09; Wilcoxon rank sum test).

In conclusion, it was found that prolonged (2 hrs) exposure to nbGL before bedtime improves overall sleep behavior, potentially through facilitation of neurons containing sleep-promoting neuropeptides.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
administering, to a subject not indicated for treatment of photophobia, green light having a characteristic wavelength only in a wavelength range between 510 nm and 550 nm with a bandwidth no larger than 20 nm full-width-half-maximum within 3 hours before the subject goes to sleep.

2. The method of claim 1, comprising extinguishing the green light after the subject goes to sleep.

3. The method of any one of claim 1 or 2, comprising continuing to administer the green light after the subject goes to sleep.

4. The method of any one of claims 1-3, wherein the subject is diagnosed with insomnia.

5. The method of any one of claims 1-4, wherein the subject has jet lag.

6. The method of any one of claims 1-5, wherein the subject is not indicated for treatment of migraine.

7. The method of any one of claims 1-6, comprising continuing to administer the green light for at least 2 hours after the subject goes to sleep.

8. The method of any one of claims 1-7, wherein at least 80% of light reaching the subject is the green light.

9. The method of any one of claims 1-8, wherein at least 90% of light reaching the subject is the green light.

10. The method of any one of claims 1-9, wherein the subject is in a darkened room.

11. The method of any one of claims 1-10, comprising administering the green light to the subject after sunset.

12. The method of any one of claims 1-11, wherein during administration of the green light, the subject is not exposed to light from a light source that does not produce the green light.

13. The method of any one of claims 1-12, wherein the subject does not use a device having a screen during the administration of the green light.

14. The method of any one of claims 1-13, wherein the subject does not use a cellphone during the administration of the green light.

15. The method of any one of claims 1-14, wherein the subject does not use a computer during the administration of the green light.

16. The method of any one of claims 1-15, wherein the subject does not use a television during the administration of the green light.

17. The method of any one of claims 1-16, comprising administering the light for at least 30 min.

18. The method of any one of claims 1-17, comprising administering the light for at least 1 hour.

19. The method of any one of claims 1-18, comprising administering the light for no more than 8 hours.

20. The method of any one of claims 1-19, comprising administering the green light to the subject's eyes.

21. The method of any one of claims 1-20, comprising directing the green light specifically to the subject's eyes.

22. The method of any one of claims 1-21, comprising administering the green light to the subject using a light source that produces the green light.

23. The method of any one of claims 1-22, comprising administering the green light to the subject using a filter that filters light from a light source to produce the green light.

24. The method of claim 23, wherein the filter comprises glasses worn by the subject.

25. The method of claim 23, wherein the filter is attached to the light source.

26. A method, comprising:
administering, to a subject not indicated for treatment of photophobia, green light having a characteristic wavelength only in a wavelength range between 510 nm and 550 nm with a bandwidth no larger than 20 nm full-width-half-maximum after sunset.

27. The method of claim 26, wherein the subject is not indicated for treatment of migraine.

28. The method of any one of claim 26 or 27, comprising extinguishing the green light after the subject goes to sleep.

29. The method of any one of claims 26-28, comprising continuing to administer the green light after the subject goes to sleep.

30. The method of any one of claims 26-29, comprising continuing to administer the green light for at least 2 hours after the subject goes to sleep.

31. The method of any one of claims 26-30, wherein at least 80% of light reaching the subject is the green light.

32. The method of any one of claims 26-31, wherein during administration of the green light, the subject is not exposed to light from a light source that does not produce the green light.

33. The method of any one of claims 26-32, comprising administering the light for at least 30 min.

34. The method of any one of claims 26-33, comprising administering the light for at least 1 hour.

35. The method of any one of claims 26-34, comprising administering the light for no more than 8 hours.

36. The method of any one of claims 26-35, comprising administering the light for no more than 2 hours.

37. The method of any one of claims 26-36, comprising administering the green light to the subject's eyes.

38. The method of any one of claims 26-37, comprising directing the green light specifically to the subject's eyes.

39. The method of any one of claims 26-38, comprising administering the green light to the subject using a light source that produces the green light.

40. The method of any one of claims 26-39, comprising administering the green light to the subject using a filter that filters light from a light source to produce the green light.

41. The method of claim 40, wherein the filter comprises glasses worn by the subject.

42. The method of claim 40, wherein the filter is attached to the light source.

43. A method, comprising:
administering, to a subject diagnosed with insomnia, green light having a characteristic wavelength only in a wavelength range between 510 nm and 550 nm with a bandwidth no larger than 20 nm full-width half-maximum within 3 hours before the subject goes to sleep.

44. The method of claim 43, wherein the subject is not indicated for treatment of photophobia.

45. The method of any one of claim 43 or 44, wherein the subject is not indicated for treatment of migraine.

46. The method of any one of claims 43-45, comprising extinguishing the green light after the subject goes to sleep.

47. The method of any one of claims 43-46, comprising continuing to administer the green light after the subject goes to sleep.

48. The method of any one of claims 43-47, comprising continuing to administer the green light for at least 2 hours after the subject goes to sleep.

49. The method of any one of claims 43-48, wherein at least 80% of light reaching the subject is the green light.

50. The method of any one of claims 43-49, wherein during administration of the green light, the subject is not exposed to light from a light source that does not produce the green light.

51. The method of any one of claims 43-50, comprising administering the light for at least 30 min.

52. The method of any one of claims 43-51, comprising administering the light for at least 1 hour.

53. The method of any one of claims 43-52, comprising administering the light for no more than 8 hours.

54. The method of any one of claims 43-53, comprising administering the green light to the subject's eyes.

55. The method of any one of claims 43-54, comprising directing the green light specifically to the subject's eyes.

56. The method of any one of claims 43-55, comprising administering the green light to the subject using a light source that produces the green light.

57. The method of any one of claims 43-56, comprising administering the green light to the subject using a filter that filters light from a light source to produce the green light.

58. The method of claim 57, wherein the filter comprises glasses worn by the subject.

59. The method of claim 57, wherein the filter is attached to the light source.

60. A method, comprising:
administering, to a subject suffering from jet lag, green light having a characteristic wavelength only in a wavelength range between 510 nm and 550 nm with a bandwidth no larger than 20 nm full-width half-maximum within 3 hours before the subject goes to sleep.

61. The method of claim 60, wherein the subject is not indicated for treatment of photophobia.

62. The method of any one of claim 60 or 61, wherein the subject is not indicated for treatment of migraine.

63. The method of any one of claims 60-62, comprising extinguishing the green light after the subject goes to sleep.

64. The method of any one of claims 60-63, comprising continuing to administer the green light after the subject goes to sleep.

65. The method of any one of claims 60-64, comprising continuing to administer the green light for at least 2 hours after the subject goes to sleep.

66. The method of any one of claims 60-65, wherein at least 80% of light reaching the subject is the green light.

67. The method of any one of claims 60-66, wherein during administration of the green light, the subject is not exposed to light from a light source that does not produce the green light.

68. The method of any one of claims 60-67, comprising administering the light for at least 30 min.

69. The method of any one of claims 60-68, comprising administering the light for at least 1 hour.

70. The method of any one of claims 60-69, comprising administering the light for no more than 8 hours.

71. The method of any one of claims 60-70, comprising administering the green light to the subject's eyes.

72. The method of any one of claims 60-71, comprising directing the green light specifically to the subject's eyes.

73. The method of any one of claims 60-72, comprising administering the green light to the subject using a light source that produces the green light.

74. The method of any one of claims 60-73, comprising administering the green light to the subject using a filter that filters light from a light source to produce the green light.

75. The method of claim 74, wherein the filter comprises glasses worn by the subject.

76. The method of claim 74, wherein the filter is attached to the light source.

77. A method, comprising:
administering, to a subject diagnosed with sleep apnea, green light having a characteristic wavelength only in a wavelength range between 510 nm and 550 nm with a bandwidth no larger than 20 nm full-width half-maximum within 3 hours before the subject goes to sleep.

78. The method of claim 77, wherein the subject is not indicated for treatment of photophobia.

79. The method of any one of claims 77-78, wherein the subject is not indicated for treatment of migraine.

80. The method of any one of claims 77-79, comprising extinguishing the green light after the subject goes to sleep.

81. The method of any one of claims 77-80, comprising continuing to administer the green light after the subject goes to sleep.

82. The method of any one of claims 77-81, comprising continuing to administer the green light for at least 2 hours after the subject goes to sleep.

83. The method of any one of claims 77-82, wherein at least 80% of light reaching the subject is the green light.

84. The method of any one of claims 77-83, wherein during administration of the green light, the subject is not exposed to light from a light source that does not produce the green light.

85. The method of any one of claims 77-84, comprising administering the light for at least 30 min.

86. The method of any one of claims 77-85, comprising administering the light for at least 1 hour.

87. The method of any one of claims 77-86, comprising administering the light for no more than 8 hours.

88. The method of any one of claims 77-87, comprising administering the green light to the subject's eyes.

89. The method of any one of claims 77-88, comprising directing the green light specifically to the subject's eyes.

90. The method of any one of claims 77-89, comprising administering the green light to the subject using a light source that produces the green light.

91. The method of any one of claims 77-90, comprising administering the green light to the subject using a filter that filters light from a light source to produce the green light.

92. The method of claim 91, wherein the filter comprises glasses worn by the subject.

93. The method of claim 91, wherein the filter is attached to the light source.

94. A method, comprising:
administering, to a subject not indicated for treatment of photophobia, green light having a characteristic wavelength only in a wavelength range between 510 nm and 550 nm with a bandwidth no larger than 20 nm full-width half-maximum and a luminance of no more than 100 cd/m².

95. The method of claim 94, wherein the green light has a luminance of no more than 75 cd/m².

96. The method of any one of claim 94 or 95, wherein the green light has a luminance of no more than 50 cd/m².

97. The method of any one of claims 94-96, wherein the green light has a luminance of no more than 25 cd/m².

98. The method of any one of claims 94-97, wherein the green light has a luminance of no more than 10 cd/m².

99. The method of any one of claims 94-98, wherein the green light has a luminance of no more than 5 cd/m².

100. The method of any one of claims 94-99, wherein the green light has a luminance of no more than 1 cd/m².

101. The method of any one of claims 94-100, wherein the subject is diagnosed as having an anxiety disorder.

102. The method of any one of claims 94-101, wherein the subject is not indicated for treatment of migraine.

103. The method of any one of claims 94-102, wherein at least 80% of light reaching the subject is the green light.

104. The method of any one of claims 94-103, wherein at least 90% of light reaching the subject is the green light.

105. The method of any one of claims 94-104, wherein the subject is in a darkened room.

106. The method of any one of claims 94-105, wherein during administration of the green light, the subject is not exposed to light from a light source that does not produce the green light.

107. The method of any one of claims 94-106, wherein the subject does not use a screen device during the administration of the green light.

108. The method of any one of claims 94-107, wherein the subject does not use a cellphone during the administration of the green light.

109. The method of any one of claims 94-108, wherein the subject does not use a computer during the administration of the green light.

110. The method of any one of claims 94-109, wherein the subject does not use a television during the administration of the green light.

111. The method of any one of claims 94-110, comprising administering the light for at least 30 min.

112. The method of any one of claims 94-111, comprising administering the light for at least 1 hour.

113. The method of any one of claims 94-112, comprising administering the light for no more than 8 hours.

114. The method of any one of claims 94-113, comprising administering the light for no more than 2 hours.

115. The method of any one of claims 94-114, comprising administering the green light to the subject's eyes.

116. The method of any one of claims 94-115, comprising directing the green light specifically to the subject's eyes.

117. The method of any one of claims 94-116, comprising administering the green light to the subject using a light source that produces the green light.

118. The method of any one of claims 94-117, comprising administering the green light to the subject using a filter that filters light from a light source to produce the green light.

119. The method of claim 118, wherein the filter comprises glasses worn by the subject.

120. The method of claim 118, wherein the filter is attached to the light source.

121. A method, comprising:
administering, to a subject having an anxiety disorder, green light having a characteristic wavelength only in a wavelength range between 510 nm and 550 nm with a bandwidth no larger than 20 nm full-width half-maximum and a luminance of no more than 100 cd/m².

122. The method of claim 121, wherein the green light has a luminance of no more than 75 cd/m².

123. The method of any one of claim 121 or 122, wherein the green light has a luminance of no more than 50 cd/m².

124. The method of any one of claims 121-123, wherein the green light has a luminance of no more than 25 cd/m².

125. The method of any one of claims 121-124, wherein the green light has a luminance of no more than 10 cd/m².

126. The method of any one of claims 121-125, wherein the green light has a luminance of no more than 5 cd/m².

127. The method of any one of claims 121-126, wherein the green light has a luminance of no more than 1 cd/m².

128. The method of any one of claims 121-127, wherein the subject is not indicated for treatment of photophobia.

129. The method of any one of claims 121-128, wherein the subject is not indicated for treatment of migraine.

130. The method of any one of claims 121-129, wherein at least 80% of light reaching the subject is the green light.

131. The method of any one of claims 121-130, wherein the subject is in a darkened room.

132. The method of any one of claims 121-131, wherein during administration of the green light, the subject is not exposed to light from a light source that does not produce the green light.

133. The method of any one of claims 121-132, comprising administering the light for at least 30 min.

134. The method of any one of claims 121-133, comprising administering the light for at least 1 hour.

135. The method of any one of claims 121-134, comprising administering the light for no more than 8 hours.

136. The method of any one of claims 121-135, comprising administering the light for no more than 2 hours.

137. The method of any one of claims 121-136, comprising administering the green light to the subject's eyes.

138. The method of any one of claims 121-137, comprising directing the green light specifically to the subject's eyes.

139. The method of any one of claims 121-138, comprising administering the green light to the subject using a light source that produces the green light.

140. The method of any one of claims 121-139, comprising administering the green light to the subject using a filter that filters light from a light source to produce the green light.

141. The method of claim 140, wherein the filter comprises glasses worn by the subject.

142. The method of claim 140, wherein the filter is attached to the light source.
